(19) United States
(12) Patent Application Publication
(10) Pub. No.: US 2007/0282042 A1
(43) Pub. Date: Dec. 6, 2007

(54) Esters of anhydrosugar alcohols as plasticizers

(76) Inventor: Anthony East, Madison, NJ (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/445,027

(22) Filed: Jun. 1, 2006

(51) Int. Cl. C08K 5/15; C08K 5/10; C07C 69/76; C08K 5/1545; C07D 323/02

(52) U.S. Cl. 524/110; 524/310; 560/112; 549/417; 549/435

(57) ABSTRACT

The invention consists of esters of anhydrosugar alcohols useful as plasticizers for polymeric compounds. Specifically, esters are formed of bis-anhydrohexitols, such as isosorbide, isomannide and isosiditide, and used as substitutes for the phthalate-based plasticizers in common use. The bis-anhydrohexitols are preferably produced from biological sources.

### STATEMENT OF GOVERNMENT INTEREST

The United States Government has rights in this application based on DOE Award No. DE-FC36-03GO13000.

### BACKGROUND OF THE INVENTION

1. Field of the Invention

The present invention relates to plasticizer compositions based on esters of anhydrosugar alcohols and, more particularly, to a bis-anhydrohexitol ester-based plasticizer composition used as a plasticizer when processing polyvinyl chloride resin.

2. Description of the Related Art

Polyvinyl chloride (PVC) is a general resin that can attain various physical processing properties by suitably mixing additives such as stabilizers, fillers, pigments, and plasticizers. Polyvinyl chloride with the various physical processing properties is widely used as a material for goods such as wallpaper, gloves, and toys, as well as for pipe, guttering, vinyl siding, electric wire insulation, and artificial leather. Plasticizers are plastic additives, most commonly phthalate esters, that give hard plastics like PVC the desired flexibility and durability. Most plasticizers are nonvolatile organic liquids or low-melting point solids, which function by reducing the normal intermolecular forces in a resin, thus permitting the macromolecular chains to slide over one another more freely. They are often based on esters of polybasic carboxylic acids with linear or branched aliphatic alcohols of moderate chain length. Plasticizers work by embedding themselves between the chains of polymers, thus spacing them apart (increasing the “molecular free volume”), and significantly lowering the glass/rubber transition temperature (Tg) for the plastic and making it softer. Some plasticizers evaporate and tend to concentrate in an enclosed space; certain plasticizers evaporating from the car interior cause the “new car smell”.

Representative plasticizers used in processing of polyvinyl chloride resin include phthalate-based plasticizers, adipate-based plasticizers, and trimellitate-based plasticizers, etc. Phthalic acid diesters, which are also called phthalates, are the primary plasticizers for most flexible polymer products, especially polymer products formed from polyvinyl chloride (PVC) and other vinyl polymers. Examples of common phthalate plasticizers include, for example, di-isononyl phthalate (DINP) and di-2-ethylhexyl-phthalate (DEHP), often called “dioctyl phthalate”. The most commonly used plasticizer, DEHP, is a phthalate, and it frequently plays the role of a standard plasticizer against which other plasticizers are evaluated.

Although phthalate plasticizers have been tested for more than 40 years and are among the most studied and best understood compounds in the world from a health and environmental perspective, phthalate plasticizers have recently come under intense scrutiny by public interest groups that are concerned about the potential of adverse health effects in children exposed to these chemicals. Consequently, there is a demand for phthalate-free plasticizers that provide the same properties when added to polymer resins such as, for example, vinyl polymers, rubbers, polyurethanes, and acrylics. Safer plasticizers with better biodegradability and less biochemical effects are being developed. Some such plasticizers include acetylated monoglycerides that are used as food additives and alkyl citrates, used in food packaging, medical products, cosmetics, and children toys.

### SUMMARY OF THE INVENTION

The invention consists of esters of anhydrosugar alcohols useful as plasticizers for polymers, particularly polyvinyl chloride. Preferably, the esters are formed of bis-anhydrohexitols and most preferably, isosorbide. Isosorbide is a 1,5:3,6 bis-anhydrohexitol which may be manufactured from biomass materials, including corn, and as such is viewed to be a “renewable resource.” The esters of the anhydrosugar are formed by reacting a suitable acid or its derivatives with the anhydrosugar in the presence of suitable solvents and catalysts, under ester-forming conditions. For example, the free acid may be reacted with the anhydrosugar in the presence of an acidic catalyst such as sulfuric acid, methanesulfonic acid, toluene para-sulfonic acid or the like, and the water produced removed by azeotropic distillation, for example with a solvent such as toluene or 1,2 dichlorethane. The acid can also be reacted with the anhydrosugar in the presence of a strong dehydrating agent such as a carbodiimide, such as dicyclohexyl-carbodiimide, or phosphorus oxychloride. Alternatively, a lower alkyl ester (methyl, ethyl, propyl or butyl ester, C₁ to C₄ carbon) is reacted in excess with the anhydrosugar in the presence of an ester-exchange catalyst, such as zinc acetate, manganese acetate, a titanium tetraalkoxide (TiOR₄ where R is preferably C₁ to C₄). The acyl halide of a suitable acid may be reacted, often in slight excess, with the anhydrosugar in the presence of a suitable solvent in the presence of an acid acceptor such as pyridine, triethylamine, or the like. The acid acceptor (e.g. pyridine) can be used as a reaction solvent. In the case of a dianhydrosugar alcohol, the acyl halide reacts at both reactive sites to produce the bis compound.

### BRIEF DESCRIPTION OF THE DRAWINGS

FIG. 1 is a drawing of the scheme of synthesis of isosorbide esters from the acid chloride of 4-(2-ethylhexyl-1-oxy)-benzoic acid, known as 4-(2-ethylhexyl-1-oxy)-benzoyl chloride, synthesized as indicated from methyl 4-hydroxy benzoate. The acid chloride is then reacted with the bisanhydrohexitol in a solvent in the presence of a suitable acid-acceptor as aforementioned.

FIG. 2 is a drawing of an alternative scheme of synthesis of isosorbide esters using an ester exchange reaction with the methyl ester product.

FIG. 3 is a drawing of an alternative scheme of synthesis wherein the plasticizer is formed by reacting the free carboxylic of a substituted benzoic acid with a bis-anhydrohexitol in the presence of a strongly acidic catalyst and optionally a suitable solvent to remove the water formed in the esterification reaction by azeotropic distillation.

### DETAILED DESCRIPTION OF A PREFERRED EMBODIMENT

As used in this specification, a sugar alcohol is a hexose that has been reduced to a hexitol. Anhydrosugar alcohols are hexitols that have been dehydrated, losing one or two molecules of water forming either one or two internal ether groups, respectively. Bis-anhydrohexitols are six-carbon sugars from which two molecules of water have been removed and retaining two alcohol moieties. Isosorbide, isomannide and isoiditide are examples of bis-anhydrohexitols. To form esters of bisanhydrohexitols it is necessary to react the compounds in question with either a carboxylic acid or a reactive derivative such as a lower alkyl ester (methyl, ethyl) or, preferably, an acyl halide. Acyl halides have the general formula RCOX where R is any alkyl or aryl group and X is a halide, preferably chloride or bromide. They are highly reactive intermediates and esterification takes place at low temperatures under mild conditions. Typical acyl halides include ethanoyl chloride, propanoyl chloride, benzoyl chloride, and the like. Particularly useful for preferred embodiments of this invention are acyl halides derived from alkyl benzoic acids, alkoxy benzoic acids, alkyl substituted benzoic acids or alkoxy substituted benzoic acids, whether 1,2-; 1,3-; or 1,4-monosubstituted or bearing more than one side chain substituent group.

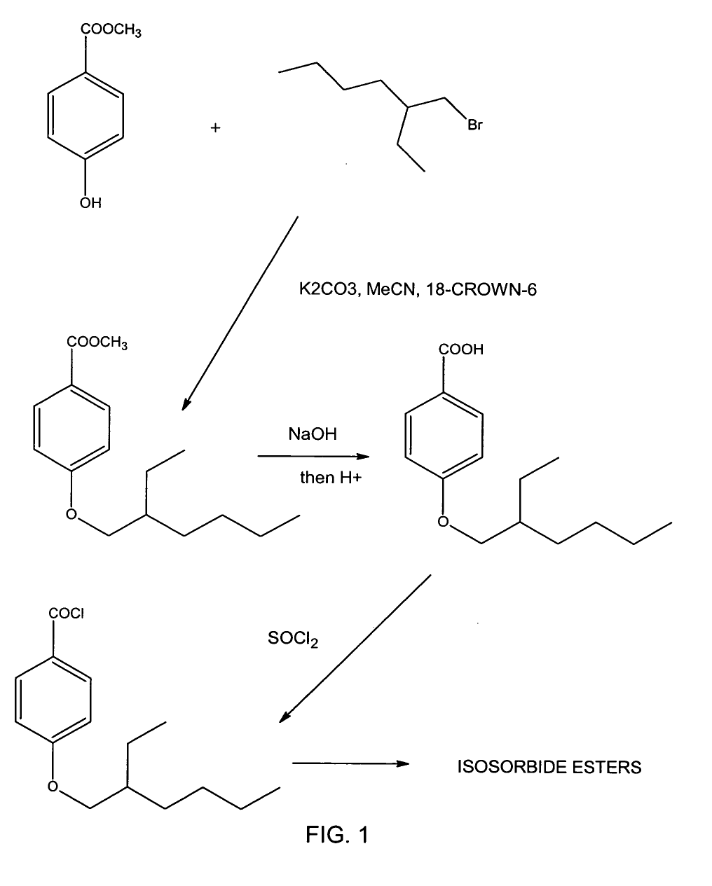

As shown in FIG. 1, the plasticizer may be formed by reacting the acid halide of a substituted benzoic acid with a bis-anhydrohexitol in the presence of an acid acceptor and optionally a suitable solvent. Examples of an acid acceptor include tertiary amines [R₃N] such as triethylamine, tributylamine, N-methyldiisopropylamine, NN-dimethylaniline, N-methylmorpholine, N-methylpiperidine, pyridine, 2,6-lutidine, and the like. Suitable solvents include chlorinated hydrocarbons such as dichloromethane, chloroform, 1,1,1-trichlorethane, 1,2-dichloroethane, tetrachloroethylene, and the like, as well as ethers such as diethyl ether, dibutyl ether, tetrahydrofuran, 1,2-dimethoxyethane (glyme), diethylene glycol dimethyl ether (diglyme), anisole, and the like. Certain hydrocarbon solvents may be also used such as benzene, toluene, cyclohexane, and the like. The amine hydrochloride salt formed as a byproduct is usually insoluble in such solvents and may be removed by filtration, and the product may be isolated by distilling off the solvent.

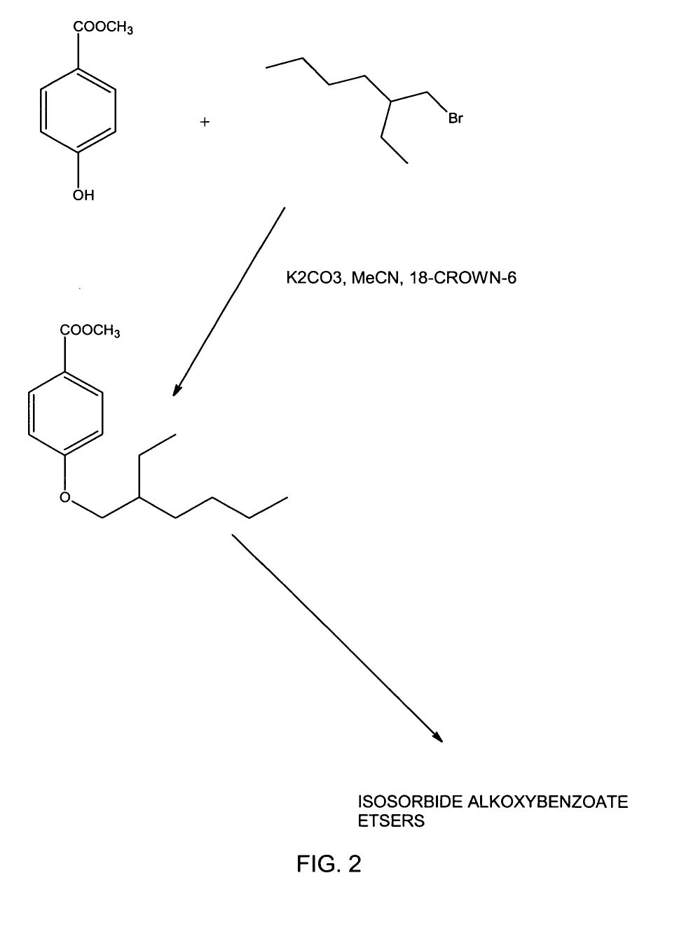

The bis-anhydrohexitol may also be reacted with an excess of methyl or ethyl ester of one of the substituted benzoic acids in the presence of an ester-exchange catalyst. Upon being heated, the lower alcohol is evolved and removed by distillation, preferably using a fractionating column to drive the chemical equilibrium and form the ester of the bisanhydrohexitol. Further distillation at higher temperatures will remove the unreacted starting materials such as the excess lower dialkyl ester. The involatile residue is the diester of the acid with the bis-anhydrohexitol. A scheme of this synthesis is depicted in FIG. 2. In a preferred embodiment, the ester exchange reaction is conducted at approximately 220° C. in the presence of a suitable catalyst, such as zinc acetate, calcium acetate, stannous 2-ethylhexanoate, dibutyltin dilaurate, manganese acetate, titanium or germanium tetra-alkoxides, wherein the alkyl group is preferably C₁ to C₄, bis-glycoloxides, or similar catalysts known to those skilled in the art, followed by distillation of the formed methanol.

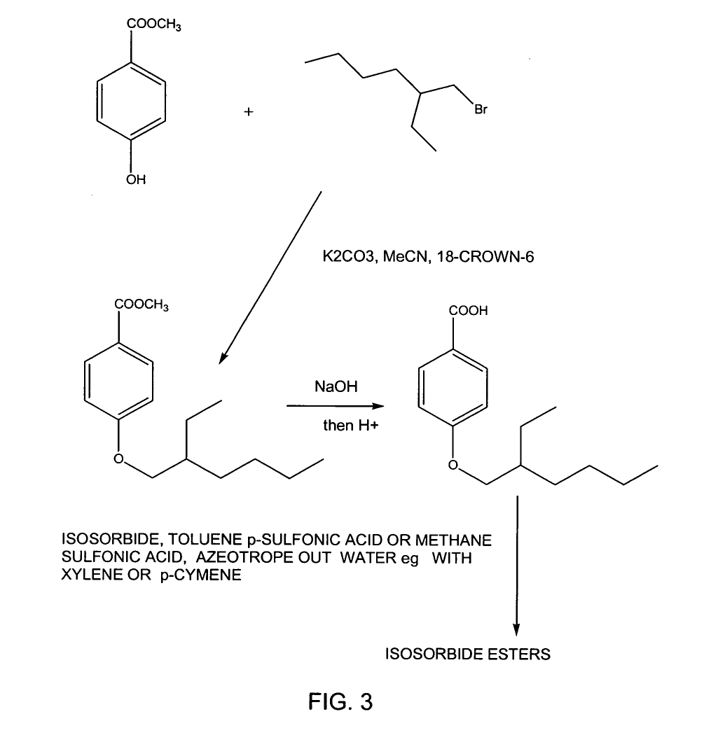

In a final method, as shown in FIG. 3, the plasticizer may be formed by reacting the free substituted benzoic acid with a bis-anhydrohexitol in the presence of an acidic ester-promoting catalyst such as a strong mineral acid, toluene-p-sulfonic acid, methanesulfonic acid, or the like, and removing the water formed continuously by azeotropic distillation with a suitable solvent such as chloroform, 1,2-dichloroethane, benzene, toluene, xylene, p-cymene and the like.

### Example 1

### Isosorbide bis4-(2-Ethylhexyloxy)Benzoate

### Preparation of Methyl4-(2-Ethylhexyloxy)Benzoate

A 1000 ml 4-necked flask was fitted with a reflux condenser, paddle stirrer and shaft seal, a pressure-equalizing tap funnel and argon gas inlet. A bubbler tube was fitted to the top of the condenser. The flask was charged with 68.4 g (0.45 moles) of methyl 4-hydroxy-benzoate, 1.0 g 18-crown-6 ether, 500 ml acetonitrile (HPLC grade) and 100 g, (0.72 moles) anhydrous potassium carbonate. The tap funnel was charged with 100 g, (0.52 mole) of 2-ethylhexyl bromide and the apparatus sparged with argon. A slow stream of argon was passed through the reaction mixture. The mixture was stirred briskly and gently refluxed while the alkyl bromide was added dropwise over 1-2 hours. Then the mixture was refluxed overnight for a total of about 20 hours and finally left to cool with stirring so that the solids did not cake in the flask. While still warm the slurry was poured into 2000 ml distilled water and stirred briskly in a 4-liter beaker. The product separated as an oil which formed an upper layer. The final pH of the aqueous layer was 9.0. The oil was separated and the aqueous layer extracted with chloroform (lower layer) to remove final traces of product. The organic phases were combined and shaken twice with water, then dried over anhydrous magnesium sulfate. Filtering and evaporation of the solvent gave the crude ester as a pale straw-colored oil weighing 116.8 g, (98% theory).

### Preparation of 4-(2-Ethylhexyloxy)-Benzoic Acid

A mixture of 116 g crude methyl ester (0.44 moles), 40 g sodium hydroxide (1.00 mole) 200 ml ethanol and 800 ml distilled water was stirred and refluxed for 2-3 hours under an argon atmosphere until a clear solution formed. The mixture was let stand overnight and next day acidified by adding a mixture of 150 ml concentrated hydrochloric acid in 200 ml distilled water. A milky emulsion formed and the product was extracted with toluene (3×200 ml) and the combined toluene layers shaken with 10% brine solution and dried overnight over anhydrous magnesium sulfate. Filtering and evaporation of the toluene gave the crude acid as a heavy oil which crystallized on standing to a white waxy solid with a melting point of 61° C. The yield was 89.8 g (82% theory).

### Preparation of 4-(2-Ethylhexyloxy)Benzoyl chloride

The crude acid from the previous experiment (89 g, 0.356 mole) was refluxed gently in thoroughly dry apparatus with 119 g (73 ml, 1.0 mole) pure colorless thionyl chloride and one drop of dry dimethylformamide as a catalyst. A gas absorption trap was fitted to the top of the condenser via a guard tube filled with Drierite to remove the hydrogen chloride and sulfur dioxide gases produced. The mixture began to react in the cold and was gently heated to reflux for 3-4 hours, by which time no white fumes of ammonium chloride were present at the top of the condenser when the gas trap was temporally removed and a strong solution of ammonia was held near the open condenser, showing that no more HCl was being produced. The excess thionyl chloride was distilled off at atmospheric pressure and final traces removed under reduced pressure. The residue was a yellow oil weighing 93 g (97% theory).

### Example 2

### Preparation of Isosorbide bis4-(2-Ethylhexyloxy)Benzoate Using the Acid Halide as Synthesized in Example 1

A 500 ml 4-neck flask was fitted with a paddle stirrer, pressure-equalizing dropping funnel and a long thermometer dipping into the liquid in the flask. The fourth neck was fitted with a short air condenser and a Drierite guard tube. All glassware was thoroughly dry. The flask was charged with 82 g (1.038 mole) of dry pyridine, 16.83 g (0.115 mole) anhydrous isosorbide, and the flask chilled in a bath of ice and salt to reduce the batch temperature to 0-5° C. A solution of 92.7 g (0.346 mole) acid chloride (prepared as above) in 100 ml dry dichloromethane was added dropwise from the tap funnel at such a rate as to keep the batch below 5° C. Gradually, the batch became cloudy and the flask filled with precipitated pyridine hydrochloride salt. When all the acid chloride had been added, the pasty mixture was left to stir overnight and warm up to room temperature. The next day, the mixture was added to 500 ml distilled water, with brisk stirring, and acidified to pH <2.0 with 50% hydrochloric acid. The lower dichloromethane layer was separated off and the acidic aqueous layer extracted twice more with dichloromethane. The combined organic phases were shaken twice with 10% sodium bicarbonate solution to remove traces of acid and once more with 10% brine solution, then dried over anhydrous magnesium sulfate. After filtration, the solvent was stripped off on the rotary evaporator to leave a viscous pale brown oil, weighing 69 g (98% theory). After a prolonged period of standing, this oil eventually crystallized to a low melting solid.

### Example 3

### Preparation of Isosorbide 2,5-bis[4-(2-Ethylhexyl)oxybenzoate]Ester by Direct Esterification

A mixture of 73 g (0.50 moles) of isosorbide, 270 g (1.08 moles) 4-(2-ethylhexyl)-oxybenzoic acid, 200 ml xylene and 1.0 g methanesulfonic acid is boiled briskly overnight under a slow nitrogen stream, under a Dean-Stark water separator until no more water collected in the Dean-Stark tube. The final yield of water is expected to be close to but less than 18.0 ml (100% theoretical). The xylene solution is shaken with three 80 ml portions of 10% sodium bicarbonate solution to remove unreacted acid and catalyst and then washed twice with 10% brine and finally with distilled water. The xylene layer is dried over anhydrous magnesium sulfate overnight, then filtered and the solvent evaporated to give a heavy brown oily liquid that is expected to yield between about 260 g and 320 g (85-95% theoretical).

### Example 4

### Measurement of the Plasticizer Effect on PVC Using a DSC Method

The DSC instrument used was a TA Instruments Model Q100 Differential Scanning Calorimeter.

Both the controls and the experimental plasticizers were run in exactly the same way. Firstly a DSC scan of commercial unplasticized powdered PVC polymer was run on a 5 mg sample using a heating rate of 10° C./minute. The temperature was taken up to about 110° C., cooled quickly and the scan repeated. There was a definite point of inflection in the second DSC trace around 73-83° C. and the mid-point of the inflection (78° C.) was taken as the Tg of unplasticized PVC. This is in agreement with literature values.

The plasticizers were mixed as follows. A 0.5 g sample of the PVC powder was mixed with 0.1 g plasticizer and the mixture ground by hand for several minutes in a small agate mortar using an agate pestle to roughly mix the two. Then a 5 mg sample of the dry mixture was taken and placed in an aluminium DSC pan, sealed, and the mixture heated to 100° C., held there for several minutes and cooled quickly. The heating and cooling cycle was repeated five times to blend the components thoroughly. The DSC was then run on the blend as before—this time the Tg was distinctly depressed to the region of 30-32° C. Several experimental plasticizers, such as various isosorbide esters, isosorbide dibenzyl ether and a dioctyl phthalate control were used. Each of the plasticizers tested depressed the Tg in the range of about 30-32° C.

Unlike isomannide and isoiditide, isosorbide is asymmetric insofar that it has non-equivalent 2-position and 5-position alcoholic moieties, one of which is known to be more reactive than the other. Hence, three different reaction products are possible, the bis-ester product, the monoester at the 2-position, and the monoester at the 5-position. With isosorbide, this gives a unique ability to tailor the groups substituted at the 2 and 5 positions which may be same or different so as to manipulate the properties of the plasticizer, for example to increase or reduce its mobility, volatility and its crystallization point. An example of the asymmetric synthesis to synthesize a mono-substituted compound, namely isosorbide-5-nitrate, is described in U.S. Pat. No. 4,297,286. The synthesis starts with isomannide and reacts it with p-toluenesulfonic acid chloride under basic conditions to make isomannide-2-p-toluenesulfonic acid ester. This is followed by a nucleophilic displacement with sodium benzoate to make isosorbide 2-monobenzoate. Because of the Walden inversion, the 2-hydroxy is isomerized to go from isomannide to isosorbide. The next step is to nitrate the 5-hydroxy to give isosorbide 2-benzoate-5-nitrate and selectively hydrolyze off the 2-benzoate group to leave isosorbide 5-nitrate.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A plasticizer for reducing the glass transition temperature of polymeric materials, comprising an ester of an anhydrosugar alcohol.

2. A plasticizer as defined in claim 1, wherein the anhydrosugar alcohol is selected from the group consisting of isosorbide, isomannide and isoiditide.

3. A plasticizer for reducing the glass transition temperature of polymeric materials, comprising a bis-ester of a bis-anhydrohexitol.

4. A plasticizer as defined in claim 3, wherein the anhydrosugar alcohol is selected from the group consisting of isosorbide, isomannide and isoiditide.

5. A method of forming a plasticizer for reducing the glass transition temperature of a polymeric material, comprising the steps of:
(a) reacting a bis-anhydrohexitol with an excess of the methyl or ethyl ester of a substituted benzoic acid in the presence of an ester-exchange catalyst;
(b) heating the reaction mixture at a first temperature to evolve the lower alcohol and separate it from the reaction mixture; and
(c) distilling the reaction mixture at second, higher temperature to remove the excess lower alkyl ester preferably under reduced pressure.

6. The method as defined in claim 5, wherein the ester exchange catalyst is selected from the group consisting of zinc acetate, calcium acetate, stannous 2-ethylhexanoate, dibutyltin dilaurate, manganese acetate, titanium tetra-alkoxides wherein the alkyl group is selected from the group consisting of C₁ to C₄, germanium tetra-alkoxides, and bis-glycoloxides.

7. A method of forming a plasticizer for reducing the glass transition temperature of a polymeric material, comprising the steps of:
(a) reacting a bis-anhydrohexitol with the free substituted benzoic acid in the presence of an acidic ester-promoting catalyst;
(b) heating the mixture in the presence of an inert solvent to remove the water formed continuously by azeotropic distillation; and
(c) distilling the reaction mixture to remove the excess solvent preferably under reduced pressure.

8. A method as defined in claim 7, wherein the acidic ester-promoting catalyst is selected from the group consisting of a strong mineral acid, toluene-p-sulfonic acid, and methanesulfonic acid.

9. A method of forming a plasticizer for reducing the glass transition temperature of a polymeric material, comprising reacting a bis-anhydrohexitol with an excess of the acid halide of a substituted benzoic acid in the presence of an acid acceptor and optionally a suitable solvent.

10. A method as defined in claim 9, wherein the acid acceptor is selected from the group consisting of tertiary amines.

11. A method as defined in claim 9, wherein the solvent is selected from the group consisting of chlorinated hydrocarbons, ethers, and hydrocarbons.

12. A method as defined in claim 10, wherein the tertiary amine is selected from the group consisting of triethylamine, tributylamine, N-methyldiisopropylamine, NN-dimethylaniline, N-methylmorpholine, N-methylpiperidine, pyridine, and 2,6-lutidine.

13. A method as defined in claim 11, wherein the chlorinated hydrocarbon is selected from the group consisting of dichloromethane, chloroform, 1,1,1-trichlorethane, 1,2-dichloroethane, and tetrachloroethylene.

14. A method as defined in claim 11, wherein the ether is selected from the group consisting of diethyl ether, dibutyl ether, tetrahydrofuran, 1,2-dimethoxyethane (glyme), anisole and diethyleneglycol dimethyl ether (diglyme).

15. A method as defined in claim 11, wherein the hydrocarbon is selected from the group consisting of benzene, toluene, and cyclohexane.

16. A plasticizer for reducing the glass transition temperature of polymeric materials, comprising a monoester of an asymmetric bis-anhydrohexitol.

17. A plasticizer as defined in claim 16, wherein the asymmetric bis-anhydrohexitol is isosorbide.
